(19) United States
(12) Patent Application Publication
(10) Pub. No.: US 2011/0282200 A1
(43) Pub. Date: Nov. 17, 2011

(54) ACCESSORY SYSTEM FOR ULTRASONIC EQUIPMENT AND INSPECTION METHOD APPLICABLE TO THE ACCESSORY SYSTEM

(75) Inventors: Ping-Lang Yen, Taipei (TW); Dar-Ren Chen, Taipei (TW); Kun-Tu Yeh, Taipei (TW)

(73) Assignees: CHANGHUA CHRISTIAN HOSPITAL, Shanghua (TW); NATIONAL TAIWAN UNIVERSITY, Taipei (TW)

(21) Appl. No.: 12/857,834

(22) Filed: Aug. 17, 2010

(30) Foreign Application Priority Data
May 11, 2010 (TW) 99114898

(51) Int. Cl. A61B 8/14

(52) U.S. Cl. 600/438; 600/459

(57) ABSTRACT

An handheld accessory system for an ultrasonic equipment and an inspection method applicable to the accessory system. The accessory system includes a force detector and a positioning device attached to a hand-held ultrasonic probe, and a signal processing device. A user may apply the ultrasonic probe to the target tumor with a certain compression depth. A force compensation module in the signal processing device allows to make compensation due to unsteady compression depth, thereby providing for the operation of transverse palpation to detect the stiffness ratio and mobility of a target relative to its surrounding tissues, and being therefore specifically suitable for diagnosing breast tumors, as benign or malignant.

### BACKGROUND OF THE INVENTION

1. Field of the Invention

This invention relates to an accessory system for an ultrasonic equipment and an inspection method applicable to the accessory system.

2. Description of Related Art

Breast cancer remains high death rate of cancer among women, and cannot be ignored because it threatens women's health and life. However, women with cancer, if cured in time, are likely to recover their health. For example, stage 0 breast cancer patients have a 5-year overall survival rate as high as 99%, and stage 1 breast cancer patients also have a 92% 5-year overall survival rate. Early recovery of breast cancer heavily relies on periodical screening and accurate diagnosis. Many patients miss the gold period of treatment without a in-time screening or accurate diagnosis. Suspicious lesions can be discovered through the periodic breast health screening or breast cancer screening. In general, a surgeon judges a lesion as benign or malignant by viewing ultrasonic images. However, the accuracy of judging a lesion as benign or malignant simply by viewing the ultrasonic images does not satisfy a doctor.

In addition to viewing the ultrasonic images, a doctor may perform an invasive diagnosis procedure, such as biopsy, on a patient to increase the accuracy of judging a lesion as benign or malignant. Recently, non-invasive procedure, such as an elastography technique comes to the market to facilitate the performance of ultrasonic images, in order to reduce the false positive rate. The elastography technique employs RF signals to calculate strain generated by tissues. The elastography-related equipment are very expensive, and are not compatible for different brands.

Therefore, how to provide an accessory system and an inspection method applicable to the accessory system that may solve the problems of the prior art, provide the stiffness ratio and mobility of tumors relative to their surrounding tissues to a doctor as a second opinion for tumor diagnosis, simplify a diagnosis process and improve diagnosis accuracy is becoming one of the most popular issues in the art.

### SUMMARY OF THE INVENTION

In view of the above-mentioned problems of the prior art, the present invention provides an accessory system attachable to an ultrasonic equipment having a hand-held ultrasonic probe, the accessory system comprising: a force detector installable on the ultrasonic probe, for detecting a force/torque received by the ultrasonic probe, generating a force/torque signal according to the detected force/torque, and sending the force/torque signal, a positioning device installable on the ultrasonic probe, for sending a space positioning signal of the ultrasonic probe; and a signal processing device having a pre-established force compensation module, the signal processing device receiving the force/torque signal and the space positioning signal, compensating, through the force compensation module, the force/torque signal according to the pre-established force compensation module to obtain a compensated force/torque signal, and performing characteristic analysis according to the compensated force/torque signal and the space positioning signal.

In an embodiment of the present invention, the force compensation module is realized by a software mechanism; and the force compensation module assumes the force received by the ultrasonic probe to have a Gaussian distribution with respect to a region right below the ultrasonic probe as a center if the compression of the force measured by the positioning device remains a constant depth; and if the compression measured by the positioning device shows a depth variation, the force received by the ultrasonic probe is adjusted such that the force/torque signal is compensated for the depth variation.

In another embodiment of the present invention, the characteristic analysis are performed by substituting the compensated force/torque signal and the space positioning signal into a pre-established mechanical module, so as to evaluate stiffness ratio and mobility of a to-be-detected object relative to surrounding tissues of the to-be-detected object.

The inspection method comprises: (1) using the ultrasonic probe to apply a compression depth on a to-be-detected object, and controlling the ultrasonic probe to move transversely on the to-be-detected object; (2) using the force detector to detect a force/torque received by the ultrasonic probe, to send a force/torque signal according to the force/torque; (3) using the positioning device to measure the compression depth and send a space positioning signal of the ultrasonic probe; and (4) using the signal processing device to receive the force/torque signal, and the space positioning signal, to compensate, through the pre-established force compensation module, the force/torque signal to obtain a compensated force/torque signal, and to perform characteristic analysis according to the compensated force/torque signal and the space positioning signal.

Compared with the prior art, an accessory system for an ultrasonic equipment and an inspection method applicable to the accessory system may be applicable to the ultrasonic equipment for tumor diagnosis, improve the accuracy of judging tumors as benign or malignant by viewing ultrasonic images, omit the necessity of pathological outcome through biopsy, simplify the process of diagnosing tumors, and reduce the equipment cost. The present invention may also improve the diagnosis accuracy by compensating the measurement errors through a compensation mechanism.

### BRIEF DESCRIPTION OF DRAWINGS

The invention can be more fully understood by reading the following detailed description of the preferred embodiments, with reference made to the accompanying drawings, wherein:

FIG. 1A is a schematic diagram of an accessory system for an ultrasonic equipment of a first embodiment according to the present invention;

FIG. 1B is a schematic diagram of an accessory system for an ultrasonic equipment of a second embodiment according to the present invention;

FIG. 2 is a diagram illustrating the operation of an accessory system according to the present invention;

FIG. 3 is a graph showing experiment results according to the present invention; and

FIG. 4 is a flow chart of an inspection method applicable to an accessory system according to the present invention.

### DETAILED DESCRIPTION OF PREFERRED EMBODIMENTS

The following illustrative embodiments are provided to illustrate the disclosure of the present invention, these and other advantages and effects can be apparently understood by those in the art after reading the disclosure of this specification. The present invention can also be performed or applied by other different embodiments. The details of the specification may be on the basis of different points and applications, and numerous modifications and variations can be devised without departing from the spirit of the present invention.

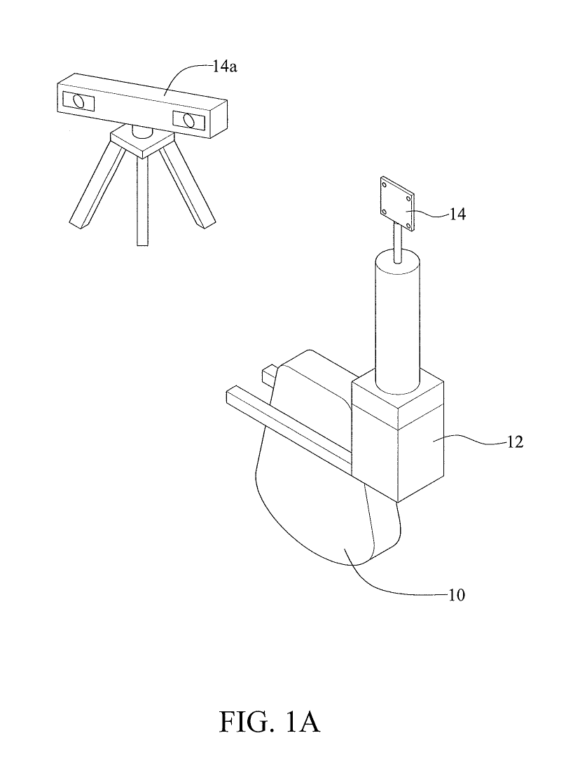

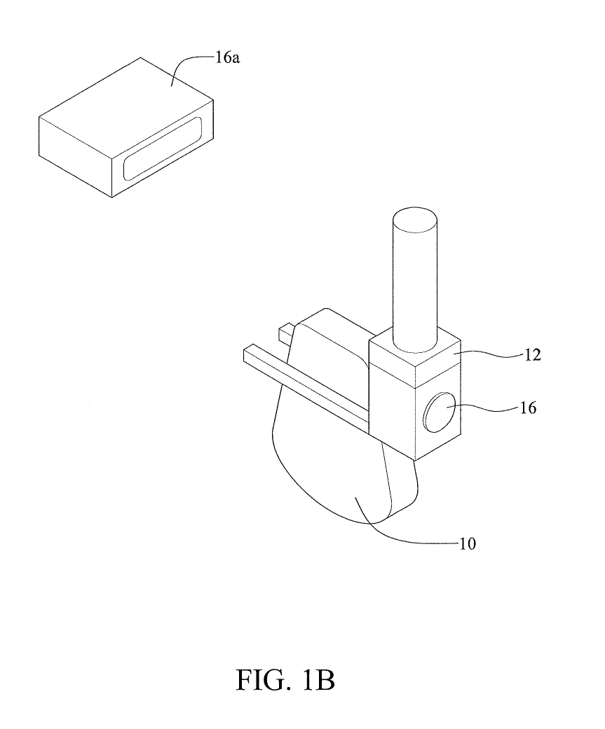

Please refer to FIG. 1A, which is a schematic diagram of an accessory system for an ultrasonic equipment of a first embodiment according to the present invention. The accessory system comprises a force detector 12, a positioning device 14 and a signal processing device (now shown). As shown in FIG. 1A, the accessory system may be attached to an ultrasonic probe 10 of the ultrasonic equipment, to facilitate tumor diagnosis. In practice, the force detector 12 and the positioning device 14 are installed on the ultrasonic probe 10.

The force detector 12 detects a force/torque received by the ultrasonic probe 10 of the ultrasonic equipment, generates a force/torque signal according to the detected force/torque, and sends the force/torque signal. In an embodiment of the present invention, the force/torque signal is in a digital or analog form.

The positioning device 14 sends a space positioning signal. In an embodiment of the present invention, the positioning device 14 is an optical positioning system, and the sent space positioning signal is an optical signal for the optical positioning system. In the first embodiment, the accessory system further comprises an optical signal receiver 14a. The optical signal receiver 14a receives the space positioning signal sent by the positioning device 14, and sends the space positioning signal to the signal processing device for further process.

The signal processing device receives, analyzes and processes the force/torque signal and the space positioning signal. In an embodiment of the present invention, the signal processing device is realized by computer software such as a personal computer program, or by a hardware circuit such as a programmed central processing unit.

In operation, when a user uses the ultrasonic probe 10, in cooperation with the accessory system of the present invention, to diagnose a disease, the measurement result may have errors because the ultrasonic probe 10 is not firmly held by the user. In order to address the problem, the signal processing device may preferably comprise a force compensation module (not shown). The force compensation module compensates the force/torque signal according to the space positioning signal to obtain a compensated force/torque signal, and performs characteristic analysis according to the compensated force/torque signal and the space positioning signal.

In an embodiment of the present invention, the force compensation module is realized by software, but is not limited thereto. The compression force of the ultrasonic probe 10 is assumed to have a Gaussian distribution with respect to a region right below the ultrasonic probe 10 as a center, the compression force is adjusted by referring to single-point downward mechanical characteristics of another region right above a to-be-detected object and normal tissues, such that the force/torque signal may be compensated. In an embodiment of the present invention, the characteristic analysis is performed by substituting the compensated force/torque signal and the space positioning signal into a pre-established mechanical module, so as to evaluate stiffness ratio and mobility of a to-be-detected object relative to its surrounding tissues. In an embodiment of the present invention, the mechanical module preferably comprises biomechanical characteristics of the to-be-detected object relative to its surrounding tissues. In an embodiment of the present invention, the to-be-detected object is preferably tumor cells, and the tumor cells are breast tumor cells, but are not limited thereto.

Therefore, the cooperation of the ultrasonic probe 10 with the accessory system of the present invention may improve the accuracy of judging a lesion as benign or malignant by viewing ultrasonic images, and simplify an examination procedure. The accessory system of the present invention may be attached to or detached from various ultrasonic equipment. For example, the accessory system of the present invention may be attached to or detached from an ultrasonic probe by a clamping, locking or adhering mechanism.

In operation, the accessory system of the present invention cooperates with the ultrasonic probe 10 only, without the necessity of performing an invasive diagnosis procedure, such that the false negative biopsy rate is reduced.

Please refer to FIG. 1B, which is a schematic diagram of an accessory system for an ultrasonic equipment of a second embodiment according to the present invention. The second embodiment differs from the first embodiment in that the positioning device 14 in the first embodiment is replaced with a positioning device 16 that may be attached to or detached from the ultrasonic probe 10 and send a space positioning signal. In an embodiment of the present invention, the positioning device 16 is an electromagnetic positioning system, and the sent space positioning signal is an electromagnetic signal for the electromagnetic positioning system. In an embodiment of the present invention, the accessory system employs an electromagnetic signal receiver 16a to replace the optical signal receiver 14a, for receiving the space positioning signal and sending the space positioning signal to the signal processing device (not shown) for further process.

In various embodiments of the present invention, the positioning device 16 may be attached to or detached from the ultrasonic probe 10 by, but not limited to, a clamping, locking, riveting, adhering and/or magnetically sucking mechanism.

The accessory system of the present invention is exemplified by the above embodiments. However, the accessory system of the present invention may have other arrangement and configurations.

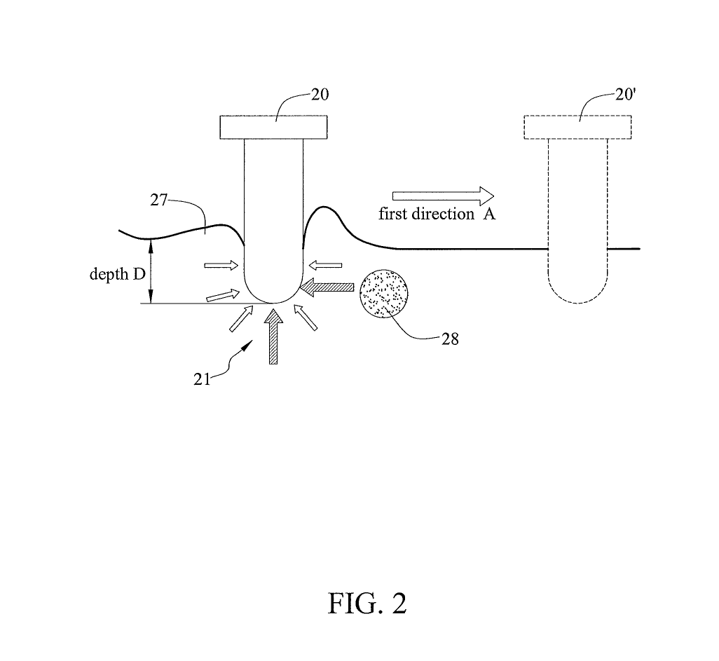

FIG. 2 shows how to use the accessory system of the present invention, in cooperation with the ultrasonic probe, to diagnose tumors. Note that the accessory system shown in FIGS. 1A and 1B may be attachable to or detachable from a ultrasonic probe 20, but is not shown in FIG. 2.

As shown in FIG. 2, a user applies a downward force to the ultrasonic probe 20 that is equipped with the accessory system, to press the ultrasonic probe 20 to a compression depth D below a normal tissue 27. A force/torque 21 that is received by the ultrasonic probe 20 at a compression depth D may be detected by the force detector 12, and a corresponding force/torque signal may be sent to the signal processing device.

The user then applies a downward force to the ultrasonic probe 20, and controls the ultrasonic probe 20 to move traverse in a first direction to arrive at a location indicated by an ultrasonic probe 20′. During the traverse movement of the ultrasonic probe 20, when the ultrasonic probe 20 moves across a tumor tissue 20, the force/torque 21 that the ultrasonic probe 20 receives may be also detected by the force detector 12. Accordingly, the force detector 12 generates the force/torque signal according to the detected force/torque, and sends the force/torque signal to the signal processing device.

Note that the force compensation module contained in the signal processing device may be realized by a software module such as a software package, or realized by a firmware module installed in the signal processing device, but is not limited thereto. Besides, the force compensation module adjusts the force/torque signal and compensates for depth variations. The operation principle of the force compensation module is described as follows. The compression force received by the ultrasonic probe is assumed to have a Gaussian distribution with respect to a region right below the ultrasonic probe as a center if the compression depth measured by the positioning device remains a constant depth. Before performing transverse palpation, two pure indentations are carried out at locations on the top of and far from the to-be-detected target. The two indentation forces are measured as:

f_{ind1}(x,z)|ₓ₌₀=a₁(e^{b}^{₁}^{z}−1), where x=0 (1)

f_{ind2}(x,z)|_{x=-∞}=a₁(e^{b}^{₂}^{z}−1), where x=−∞ (2)

Equations (1) and (2) may be employed to represent the indentation forces applied at a region right above (x=0) the to-be-detected object (e.g., tumor) and at another region (x=−∞) far from the region right above the to-be-detected object, respectively, where z represents the downward depth (z₀ represents the downward depth right below the ultrasound probe 20). Equation (1) may be combined with equation (2) to obtain equation (3) for a compression force during transverse palpation:

$${f}_{ind} ( x , z ) = α ( z ) ( {e}^{- \frac{{x}^{2}}{{σ}^{2} ( z )} z} - 1 ) + β ( z ) , \qquad ( 3 )$$

By the substitution Of boundary conditions, equations (4), (5) and (6) may be obtained as follows:

$$\begin{matrix}α ( z ) = {a}_{1} ( {e}^{{b}_{1} z} - 1 ) - {a}_{2} ( {e}^{{b}_{2} z} - 1 ) , & ( 4 ) \\ β ( z ) = {a}_{1} ( {e}^{{b}_{1} z} - 1 ) , & ( 5 ) \\ {σ}^{2} ( z ) \approx \frac{{L}^{2}}{ln ( \frac{{f}_{ind 1}}{{f}_{ind 2}} )} , & ( 6 )\end{matrix}$$

where L is a distance far enough from a region right above the to-be-detected object. Therefore, the force compensation model of equation (6) may be obtained as follows:

$${f}_{z} ( x , {z}_{0} ) \approx {f}_{z} ( x , z ) - δ {f}_{ind} - {f}_{z} ( x , z ) - \frac{\partial {f}_{ind}}{\partial z} {\vert}_{z = {z}_{0}} δ z , \qquad ( 7 )$$

where δf_{ind} is a compression force variation, and a depth variation δz is sufficiently small, which causes equation (7) to be linearly approximate.

Since normal tissues have different biomechanical characteristics from tumor tissues, whether a portion that the ultrasonic probe 20 is going to diagnose has tumor tissues may be determined by the comparison of the force/torque signal. Further, the characteristic analysis may be performed or the tumors may be classified according to various biomechanical characteristics. Therefore, a doctor may use the ultrasonic probe 20, in cooperation with the accessory system of the present invention, to diagnose tumors. Accordingly, it is convenient for the doctor to diagnose tumor tissues such as breast tumor tissues by viewing ultrasonic images, a diagnose process is simplified, and the cost is reduced.

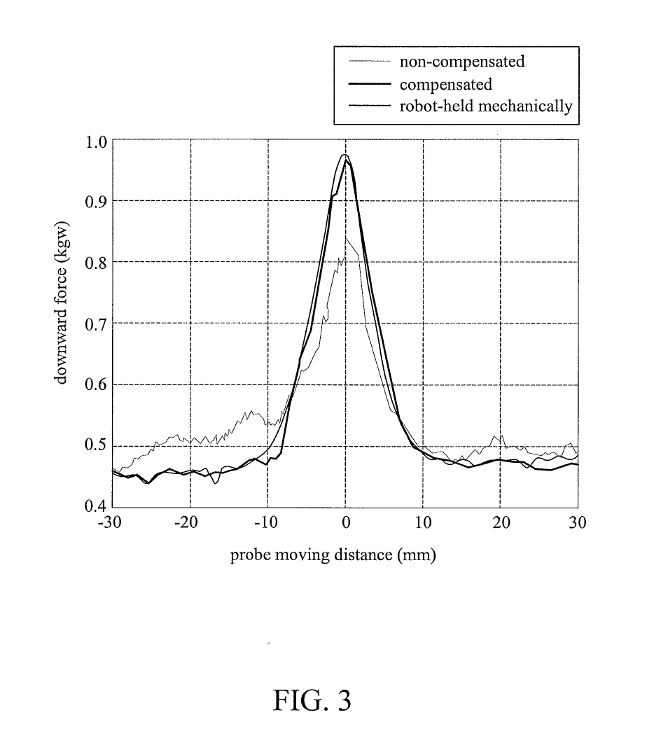

FIG. 3 shows a relation between a compression force and a probe moving distance for a robot-held probe, handheld uncompensated probe and handheld compensated probe. FIG. 3 reveals that the results of the handheld compensated probe and the robot-held are very close, and are more stable and closer to the optimal result than the result of the handheld uncompensated probe.

In conclusion, the measurement errors caused by a handheld probe may be greatly reduced by the force compensation module of the present invention that provides accurate compensation to the unstable compression depth applied by the handheld probe.

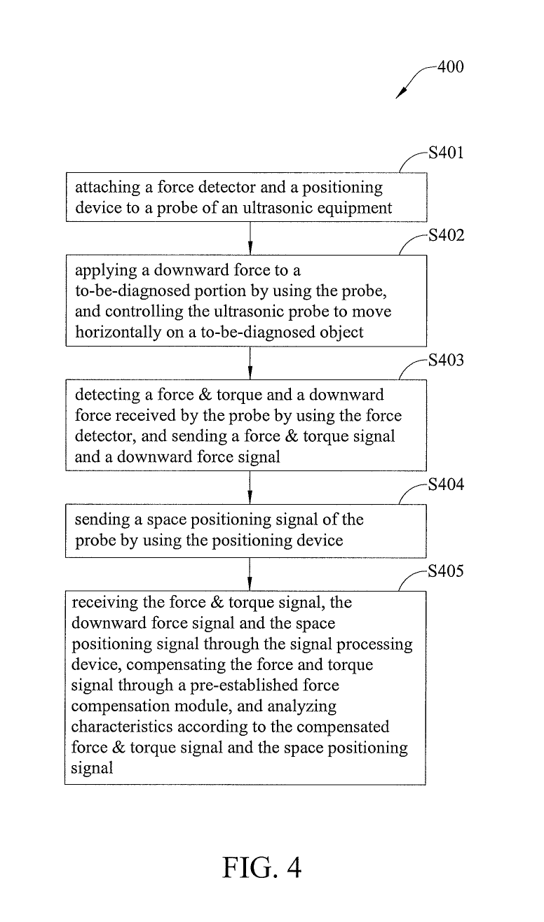

FIG. 4 is a flow chart of an inspection method 400 applicable to the accessory system of the present invention. In step S401, an ultrasonic probe is equipped with the accessory system of the present invention. Proceed to step S402.

In step S402, the ultrasonic probe applies a compression depth to the to-be-detected object, and moves on the to-be-detected object transversely. Proceed to step S403.

In step S403, the force detector detects a force/torque that the ultrasonic probe receives, sends a force/torque signal according to the force/torque. Proceed to step S404.

In step S404, the positioning device measures the compression depth and sends a space positioning signal of the ultrasonic probe. Proceed to step S405.

In step S405, the signal processing device receives the force/torque signal, the downward force signal and the space positioning signal, compensates the force/torque signal through a pre-established force compensation module to obtain a compensated force/torque signal, and performs characteristic analysis according to the compensated force/torque signal and the space positioning signal.

Compared with the prior art, an accessory system for an ultrasonic equipment and an inspection method applicable to the accessory system of the present invention may cooperate with the ultrasonic equipment to diagnose, and improve the lack of accuracy of tumor diagnose and too complicated the diagnose process through the space information and force/torque information of the measured tumor location.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. An ultrasonic-equipment accessory system attachable to an ultrasonic equipment having a hand-held ultrasonic probe, the accessory system comprising:
a force detector installable on the ultrasonic probe, for detecting a force/torque received by the ultrasonic probe, generating a force/torque signal according to the detected force/torque, and sending the force/torque signal;
a positioning device installable on the ultrasonic probe, for sending a space positioning signal of the ultrasonic probe; and
a signal processing device having a pre-established force compensation module, the signal processing device receiving the force/torque signal and the space positioning signal, compensating, through the force compensation module, the force/torque signal according to the pre-established force compensation module to obtain a compensated force/torque signal, and performing characteristic analysis according to the compensated force/torque signal and the space positioning signal.

2. The accessory system of claim 1, wherein the positioning device employs an optical positioning mechanism, and the space positioning signal is an optical signal for the optical positioning mechanism.

3. The accessory system of claim 1, wherein the positioning device employs an electromagnetic positioning mechanism, and the space positioning signal is an electromagnetic signal for the electromagnetic positioning mechanism.

4. The accessory system of claim 1, wherein the force compensation module assumes the force/torque received by the ultrasonic probe to have a Gaussian distribution with respect to a region right below the ultrasonic probe as a center if a compression of the force/torque measured by the positioning device remains a constant depth; and if the compression measured by the positioning device shows a depth variation, the force received by the ultrasonic probe is adjusted such that the force/torque signal is compensated for the depth variation.

5. The accessory system of claim 4, wherein the force compensation module is a software module or a firmware module.

6. The accessory system of claim 4, wherein the to-be-detected object is tumor cells.

7. The accessory system of claim 6, wherein the tumor cells are breast tumor cells.

8. The accessory system of claim 1, wherein the characteristic analysis is performed by substituting the compensated force/torque signal and the space positioning signal into a pre-established mechanical module, so as to evaluate stiffness ratio and mobility of a to-be-detected object relative to surrounding tissues of the to-be-detected object.

9. The accessory system of claim 1, wherein the positioning device is attachable to or detachable from the ultrasonic probe by a clamping, locking, riveting, adhering and/or magnetically sucking mechanism.

10. An inspection method applicable to the accessory system of claim 1, the method comprising steps of:
(1) using the ultrasonic probe to apply a compression depth on a to-be-detected object, and controlling the ultrasonic probe to move transversely on the to-be-detected object;
(2) using the force detector to detect a force/torque received by the ultrasonic probe to send a force/torque signal according to the force/torque;
(3) using the positioning device to measure the compression depth and send a space positioning signal of the ultrasonic probe; and
(4) using the signal processing device to receive the force/torque signal and the space positioning signal, so as to compensate, through the pre-established force compensation module, the force/torque signal to obtain a compensated force/torque signal, and to perform characteristic analysis according to the compensated force/torque signal and the space positioning signal.

11. The inspection method of claim 10, wherein in step (3), the positioning device employs an optical positioning mechanism, and the space positioning signal is an optical signal for the optical positioning mechanism.

12. The inspection method of claim 10, wherein in step (3), the positioning device employs an electromagnetic positioning mechanism, and the space positioning signal is an electromagnetic signal for the electromagnetic positioning mechanism.

13. The inspection method of claim 10, wherein the force received by the ultrasonic probe is assumed to have a Gaussian distribution with respect to a region right below the ultrasonic probe as a center if the compression depth measured by the positioning device remains a constant depth; and if the compression depth measured by the positioning device shows a depth variation, the force received by the ultrasonic probe is adjusted such that the force/torque signal is compensated for the depth variation.

14. The inspection method of claim 10, wherein in step (4), the compensated force/torque signal and the space positioning signal are substituted into a pre-established mechanical module, so as to evaluate stiffness ration and mobility of the to-be-detected object relative to surrounding tissues of the to-be-detected object.

15. The inspection method of claim 10, wherein the to-be-detected object is tumor cells.

16. The inspection method of claim 15, wherein the tumor cells are breast tumor cells.
